(19) United States
(12) Patent Application Publication
(10) Pub. No.: US 2017/0027660 A1
(43) Pub. Date: Feb. 2, 2017

(54) TAGGED SURGICAL INSTRUMENTS AND METHODS THEREFOR

(71) Applicant: COVIDIEN LP, Mansfield, MA (US)

(72) Inventor: William A. Blair, San Diego, CA (US)

(21) Appl. No.: 15/303,286

(22) PCT Filed: Oct. 4, 2015
(86) PCT No.: PCT/US2015/025376

§ 371 Date: Oct. 11, 2016

Related U.S. Application Data
(60) Provisional application No. 61/978,789, filed on Apr. 11, 2014.

(51) Int. Cl. A61B 90/00; A61B 17/06; A61B 8/08; A61B 90/90

(52) U.S. Cl. CPC A61B 90/39; A61B 90/90; A61B 17/06004; A61B 8/481; A61B 2090/3929; A61B 2090/3933; A61B 2090/3983; A61B 2017/00526

(57) ABSTRACT

Provided herein is a tag comprising an imaging sphere suspended within a matrix. In some embodiments, the matrix comprises a polymer. In some embodiments, the matrix comprises an adhesive polymer. In some embodiments, the matrix comprises a shelf-stable polymer. In some embodiments, the matrix comprises an epoxy. In some embodiments, the adhesive polymer comprises cyanoacrylate. In some embodiments, the imaging sphere comprises a cavity loaded with an imaging agent surrounded by a shell. In some embodiments, the imaging agent exhibits echogenic properties. Further provided is a method for preparing an imaging sphere comprising: depositing organic or inorganic material onto a bead surface to form a sphere with a solid core; removing the solid core by solvent extraction or calcination to form a hollow sphere; and loading the hollow sphere with an imaging agent.

### CROSS-REFERENCE TO RELATED APPLICATIONS

This application is a U.S. National Stage Application filed under 35 U.S.C. §371(a) of International Patent Application No. PCT/U52015/025376, filed Oct. 4, 2015, which claims the benefit of U.S. Provisional Application Ser. No. 61/978,789, filed on Apr. 11, 2014, which is hereby incorporated by reference in its entirety.

### SUMMARY OF THE INVENTION

Provided herein is a tag comprising an imaging sphere suspended within a matrix. In some embodiments, the matrix comprises a polymer. The polymer may comprise an adhesive polymer. The polymer may comprises cyanoacrylate. In some embodiments, the polymer comprises a shelf-stable polymer. In some embodiments, the matrix comprises an epoxy. In some embodiments, the imaging sphere comprises a cavity loaded with an imaging agent surrounded by a shell. In some embodiments, the imaging agent exhibits echogenic properties. In some embodiments, the imaging agent is a gas. In some embodiments, the gas is dissolved in a solution. In some embodiments, the gas resonates when impinged upon by ultrasound. In further embodiments, the gas emits ultrasound at harmonic frequencies. In some embodiments, the shell comprises an inorganic material. In some embodiments, the inorganic material comprises silica or titanium. In some embodiments, disclosed herein is a tagged surgical instrument comprising a surgical instrument and the tag described herein. In some embodiments, the instrument is a needle. In further embodiments, the tag is embedded in a thread filament. In other embodiments, the tag attaches a thread filament to the needle. Provided herein is a method for preparing an imaging sphere comprising: depositing organic or inorganic material onto a bead surface to form a sphere with a solid core; removing the solid core by solvent extraction or calcination to form a hollow sphere; and loading the hollow sphere with an imaging agent. In some embodiments, the inorganic material is silica or titanium. In some embodiments, the bead is a polymeric bead. In some embodiments, the polymeric bead comprises amine-functionalized polystyrene. In some embodiments, the polymeric bead comprises one or more of the following polymers: o-polyacrylamine, poly (vinyl chloride), poly (vinyl chloride) carboxylated, polystyrene, polypropylene, or poly (vinyl chloride-co-vinyl acetate co-vinyl) alcohols. In some embodiments, the bead has a diameter between about 40 nm to about 500 micron. In some embodiments, the bead has a diameter between about 400 nm to about 100 micron. In some embodiments, the bead has a diameter between about 500 nm to about 10 micron. In some embodiments, the bead has a diameter greater than 500 nm. In some embodiments, the organic or inorganic material further comprises a dopant. In some embodiments, the dopant is Iron(III) Oxide or Boron. In some embodiments, the method further comprises subjecting the hollow sphere to a vacuum prior to loading the hollow sphere with an imaging agent. In some embodiments, the imaging agent exhibits echogenic properties. In some embodiments, the imaging agent is a gas. In some embodiments, the gas is dissolved in a solution. In some embodiments, the gas resonates when impinged upon by ultrasound. In some embodiments, the gas emits ultrasound at harmonic frequencies. Provided herein is a method for tagging a surgical instrument comprising applying a tag, wherein the tag comprises an imaging sphere suspended within a matrix, to the instrument as a coating on a portion of the surgical instrument. In some embodiments, the tag comprises an adhesive polymer. Provided herein is a method for tagging a surgical instrument comprising applying a tag, wherein the tag comprises an imaging sphere suspended within a matrix, to a portion of a surface of the instrument. In some embodiments, the tag is applied to the surface as a dot or stripe.

Provided herein is a tag comprising an imaging sphere suspended within a matrix. In some embodiments, the matrix comprises a polymer. In some embodiments, the matrix comprises an adhesive polymer. In some embodiments, the matrix comprises a shelf-stable polymer. In some embodiments, the matrix comprises an epoxy. In some embodiments, the adhesive polymer comprises cyanoacrylate. In some embodiments, the imaging sphere comprises a cavity loaded with an imaging agent surrounded by a shell. In some embodiments, the imaging agent exhibits echogenic properties. In some embodiments, the imaging agent is a gas. In some embodiments, the gas is dissolved in a solution. In some embodiments, the gas resonates when impinged upon by ultrasound. In some embodiments, the gas emits ultrasound at harmonic frequencies. In some embodiments, the shell comprises an inorganic material. In some embodiments, the inorganic material is silica or titanium. Provided herein is a tagged surgical instrument comprising a surgical instrument and a tag. In some embodiments, the instrument is a needle. In some embodiments, the tag is embedded in a thread filament. In some embodiments, the tag attaches a thread filament to the needle. Provided herein is a method for preparing an imaging sphere comprising:(i) depositing organic or inorganic material onto a bead surface to form a sphere with a solid core; (ii) removing the solid core by solvent extraction or calcination to form a hollow sphere; and (iii) loading the hollow sphere with an imaging agent. In some embodiments, the inorganic material is silica or titanium. In some embodiments, the bead is a polymeric bead. In some embodiments, the polymeric bead comprises amine-functionalized polystyrene. In some embodiments, the polymeric bead comprises one or more of the following polymers: o-polyacrylamine, poly (vinyl chloride), poly (vinyl chloride) carboxylated, polystyrene, polypropylene, or poly (vinyl chloride-co-vinyl acetate co-vinyl) alcohols. In some embodiments, the bead has a diameter between about 40 nm to about 500 micron. In some embodiments, the bead has a diameter between about 400 nm to about 100 micron. In some embodiments, the bead has a diameter between about 500 nm to about 10 micron. In some embodiments, the bead has a diameter greater than 500 nm. In some embodiments, the organic or inorganic material further comprises a dopant. In some embodiments, the dopant is Iron (III) Oxide or Boron. In some embodiments, the hollow sphere is subjected to a vacuum prior to loading the hollow sphere with an imaging agent. In some embodiments, the imaging agent exhibits echogenic properties. In some embodiments, the imaging agent is a gas. In some embodiments, the gas is dissolved in a solution. In some embodiments, the gas resonates when impinged upon by ultrasound. In some embodiments, the gas emits ultrasound at harmonic frequencies. Provided herein is a method for tagging a surgical instrument comprising applying a tag to the instrument as a coating on a portion of the surgical instrument. In some embodiments, the tag comprises an adhesive polymer. Provided herein is a method for tagging a surgical instrument comprising applying a tag to a portion of a surface of the instrument. In some embodiments, the tag is applied to the surface as a dot or stripe.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying figures of which:

FIG. 1A depicts an embodiment including a surgical instrument comprising a surgical needle, a surgical thread, and a tag comprising an imaging sphere suspended in an adhesive polymer placed at the joint area of the surgical needle and surgical thread.

FIG. 1B depicts a magnified view of the joint area of the surgical instrument embodiment of FIG. 1A.

### DETAILED DESCRIPTION OF THE INVENTION

Provided herein is a tag comprising an imaging sphere suspended within a matrix.

Provided herein is a method for preparing an imaging sphere comprising: depositing organic or inorganic material onto a bead surface to form a sphere with a solid core; removing the solid core by solvent extraction or calcination to form a hollow sphere; and loading the hollow sphere with an imaging agent.

Provided herein is a method for tagging a surgical instrument comprising applying a tag, wherein the tag comprises an imaging sphere suspended within a matrix, to the instrument as a coating on a portion of the surgical instrument.

Provided herein is a method for tagging a surgical instrument comprising applying a tag, wherein the tag comprises an imaging sphere suspended within a matrix, to a portion of a surface of the instrument.

Provided herein are methods, compositions, materials, tags, and surgical instruments related to the detection of instruments before, during or after surgery. In some embodiments, the methods, compositions, materials, tags, and surgical instruments relate to providing an image or signal detecting instruments that are removed from or that have not been removed from a subject undergoing surgery.

Provided herein are methods for detecting a surgical instrument with the use of an imaging technique, for non-limiting example, an imaging technique such as ultrasound. Provided herein are surgical instruments wherein a detectable tag is present on or in the instrument. In some embodiments, a detectable tag is incorporated into the surgical instrument, for non-limiting example, in polymer thread filaments for a needle. In some embodiments, tagged surgical instruments are disposable. In some embodiments, a needle incorporates a tag into the joint area, for non-limiting example, the joint area between the metal head and the sewing thread. In some embodiments, a surgical instrument is coated with a tag. In some embodiments, a surgical instrument is tagged with a discrete dot or stamp that is detectable by an imaging technique or an imaging device while the surgical instrument is within the body of a subject. Thus, the tag is detectable at body temperature.

Provided herein are a variety of suitable tags for detecting of surgical instruments. In some embodiments, the tags comprise compositions of imaging spheres suspended in a polymer matrix. Provided herein are spheres loaded with imaging agents to be used in tags as described herein. In some embodiments, the sphere comprises a shell that is detectable by an imaging technique or an imaging device. In some embodiments, the sphere comprises a core that is detectable by an imaging technique or an imaging device. In some embodiments, the sphere comprises a shell having a core comprising an imaging agent, for non-limiting example, a gas, that is detectable by an imaging technique or an imaging device. In some embodiments, imaging spheres are suspended or embedded in a matrix, for non-limiting example, polymer matrix or epoxy. In some embodiments, an imaging agent is attached to the inner or outer surface of a sphere's shell.

Provided herein are tagged surgical instruments that are suitable for storage conditions. In some embodiments, tagged surgical instruments are stable to heat, humidity or both. In some embodiments, tagged surgical instruments exhibit shelf-stability over various lengths of time. In some embodiments, materials, compositions, and tags are suitable for storage conditions. In some embodiments, materials, compositions, and tags are stable to heat, humidity or both. In some embodiments, materials, compositions, and tags exhibit shelf-stability over various lengths of time. In some embodiments, methods are provided for increasing shelf-stability of a tagged surgical instrument, composition, tag, or material. In some embodiments, methods for increasing shelf-stability comprise doping a sphere's shell, wherein the sphere or a plurality of the spheres is incorporated into a tag, such as into a coating on a surgical instrument and wherein the sphere comprises a core that is detectable by an imaging technique or by an imaging device.

### DEFINITIONS

Unless defined otherwise, all technical and scientific terms used herein have the same meaning as commonly understood to one of ordinary skill in the art to which this disclosure belongs. It is known to one of skill in the art of surgical instruments that instruments do not follow a standardized naming convention and may be referred to by multiple names. For the sake of clarity, some surgical instruments are defined herein according to their function. However, one of skill in the art will recognize that those instruments may have alternative names.

The term “dopant” refers to a charge-transfer agent used to generate, by oxidation or reduction, positive or negative charges in an intrinsically conducting polymer. Examples of dopants include AsF₅ or I₂ as oxidizing agents, generating cation radicals on the chains of an intrinsically conducting polymer (so-called holes), or a solution of sodium naphthalenuidyl in tetrahydrofuran as a reducing agent, generating anion radicals on the chains of an intrinsically conducting polymer.

The term “calcination” refers to heating a substance to high temperatures in air or oxygen.

The term “emulsion polymerization” refers to a polymerization process whereby monomer(s), initiator, dispersion medium, and possibly colloid stabilizer initially an in homogeneous system resulting in particles of colloidal dimensions containing the formed polymer.

The term “forceps” refers to a surgical instruments used to grasp, hold or occlude. Examples include, but are not limited to, hemostats, dressing and tissue forceps, bone holding forceps, and needle holders.

The term “imaging agent” refers to a chemical substance that is capable of detection with the aid of a medical imaging device, including but not limited to, fluoroscopy, magnetic resonance imaging (MRI), ultrasound, X-ray, positron emission tomography (PET), SPECT tomography, and any compatible tomography imaging.

The term “nanosphere” refers to a sphere in the nanometer size range that ranges from about 40 nanometers in diameter to about 500 nanometers in diameter, at most about 500 nanometers in diameter, from 40 nanometers in diameter to 500 nanometers in diameter, or at most 500 nanometers in diameter. A nanosphere need not be perfectly spherical in shape, and may have an average diameter as noted herein, that is, the average diameter may be from about 40 nanometers to about 500 nanometers, at most about 500 nanometers, from 40 nanometers to 500 nanometers, or at most 500 nanometers, while other diameters of the nanosphere may be larger or smaller than this average. Further, a plurality of nanospheres used in the tags disclosed herein may not be perfectly uniform in diameter, and may include a range of average diameters, so long as the overall average diameter of the plurality of nanospheres ranges from about 40 nanometers to about 500 nanometers, at most about 500 nanometers, from 40 nanometers to 500 nanometers, or at most 500 nanometers. As used herein with respect to the size of nanospheres, the term “about” means ranges of 1%, 5%, 10%, or 25% of the diameter. When a nanosphere includes an imaging agent, it is referred to as an “imaging nanosphere” herein, and is configured to be capable of being imaged in a subject during surgery or after a surgery by an imaging technique or by an imaging device as noted herein.

The term “sphere” refers to a sphere that ranges from about 40 nanometers in diameter to about 0.5 millimeters in diameter, at most about 0.5 millimeters in diameter, from 40 nanometers in diameter to 0.5 millimeters in diameter, at most 0.5 millimeters in diameter, from about 40 nanometers in diameter to about 0.1 millimeters in diameter, at most about 0.1 millimeters in diameter, from 40 nanometers in diameter to 0.1 millimeters in diameter, or at most 0.1 millimeters in diameter. In some embodiments, a sphere comprises a diameter ranging in size from about 40 nanometers to about 500 micrometers (micron); from about 40 nanometers to about 300 micron; from about 100 nanometers to about 100 micron; from about 200 nanometers to about 1 micron; from about 200 nanometers to about 700 nanometers; or from about 400 nanometers to about 500 nanometers. A sphere need not be perfectly spherical in shape, and may have an average diameter as noted herein, that is, the average diameter may be from about 40 nanometers to about 0.5 millimeters, at most about 0.5 millimeters, from 40 nanometers to 0.5 millimeters, at most 0.5 millimeters, from about 40 nanometers to about 0.1 millimeters, at most about 0.1 millimeters, from 40 nanometers to 0.1 millimeters, at most 0.1 millimeters, about 40 nanometers to about 500 micrometers (micron), from about 40 nanometers to about 300 micron, from about 100 nanometers to about 100 micron, from about 200 nanometers to about 1 micron, from about 200 nanometers to about 700 nanometers, or from about 400 nanometers to about 500 nanometers, while other diameters of the sphere may be larger or smaller than this average. Further, a plurality of spheres used in the tags disclosed herein may not be perfectly uniform in diameter, and may include a range of average diameters, so long as the overall average diameter of the plurality of spheres ranges from about 40 nanometers to about 0.5 millimeters, at most about 0.5 millimeters, from 40 nanometers to 0.5 millimeters, at most 0.5 millimeters, from about 40 nanometers to about 0.1 millimeters, at most about 0.1 millimeters, from 40 nanometers to 0.1 millimeters, at most 0.1 millimeters, about 40 nanometers to about 500 micrometers (micron), from about 40 nanometers to about 300 micron, from about 100 nanometers to about 100 micron, from about 200 nanometers to about 1 micron, from about 200 nanometers to about 700 nanometers, or from about 400 nanometers to about 500 nanometers. As used herein with respect to the size of spheres, the term “about” means ranges of 1%, 5%, 10%, or 25% of the diameter. When a sphere includes an imaging agent, it is referred to as an “imaging sphere” herein, and is configured to be capable of being imaged in a subject during surgery or after a surgery by an imaging technique or by an imaging device as noted herein. Any of the spheres or imaging spheres described herein may be of the nano-size, without referring to them specifically as nanospheres or imaging nanospheres specifically. Thus, spheres may include nanospheres and/or larger-than-nano sized spheres; and imaging spheres may include imaging nanospheres and/or larger-than-nano sized imaging spheres.

The term “polymer bead” refers to a sphere of polymer, e.g., polystyrene beads.

The term “polymer emulsion” refers to an emulsion in which the dispersed phase is a liquid polymer or a polymer solution.

The term “porosity” refers to the pore space in a material.

The term “retractor” refers to a surgical instrument used to retract. Examples include, but are not limited to, hand-held and self-retaining retractors, as well as instruments such as skin and bone hooks.

The term “sharp” refers to a surgical instrument used to cut or incise. Examples include, but are not limited to, scissors, knives, scalpels, chisels and osteotomes, among others.

The term “support medium” refers to a lubricant or glue. Examples include, but are not limited to, silicone, rtp, cyanoacrylate and acrylic, among others.

The term “instruments” or “surgical instruments” or “surgical instrument devices” or “surgical devices” refer to the singular or plural version of any one or more of the following example surgical instruments: articulator, bone chisel, cottle cartilage crusher, bone cutter, bone distractor, Ilizarov apparatus, intramedullary kinetic bone distractor, bone drill, bone lever, bone mallet, bone rasp, bone saw, bone skid, bone splint, caliper, cannula, cautery, curette, depressor, dilator, dissecting knife, surgical Pinzette, dermatome, forceps such as bone forceps, carmalt forceps, crushing forceps, dandy forceps Debakey forceps, dissecting forceps, Doyen intestinal clamp, epilation forceps, Halstead forceps, Kelly forceps, Kocher forceps, mosquito forceps, tissue forceps, sponge forceps, Backhaus towel forceps, Lorna towel forceps, or towel forceps, acanthulus, acanthabolos, hooks such as a nerve hook, obstetrical hook, or skin hook, lancet, scalpel, luxator, lythotome, lythotript, mallet, Partsch mallet, mammotome, needle holder, occluder, ostotome, Epker osteotome, periosteal elevator, Joseph elevator, molt periosteal elevator, Obweg periosteal elevator, septum elevator, Tessier periosteal elevator, probe, retractors such as Deaver retractor, Gelpi retractor, Weitlaner retractor, USA-Army/Navy retractor, O'Connor-O'Sullivan retractor, Methieu retractor, Jackson tracheal hook, Crile retractor, Meyerding finger retractor, Little retractor, Love nerve retractor, Green retractor, Goelet retractor, Cushing vein retractor, Langenbeck retractor, Richardson retractor, Richardson-Eastmann retractor, Kelly retractor, Parker retractor, Parker-Mott retractor, Roux retractor, Mayo-Collins retractor, ribbon retractor, Alm retractor, self-retaining retractors, Beckman-Weitlaner retractor, Beckman-Eaton retractor, Beckman retractor, or Adson retractor, rib spreader, rongeur, ultrasonic scalpel, laser scalpel, scissors, iris scissors, Kiene scissors, Metzenbaum scissors, Mayo scissors, Tenotomy scissors, spatula, speculum, mouth speculum, rectal speculum, Sim's vaginal speculum, Cusco's vaginal speculum, sponge bowl, sterilization tray, sterna saw, suction tube, surgical elevator, surgical hook, surgical knife, surgical mesh, surgical needle, surgical snare, surgical sponge, surgical spoon, surgical stapler, surgical tray, suture, tongue depressor, tonsillotome, towel clamp, tracheotome, tissue expander, subcutaneous inflatable balloon expander, trephine, and trocar. Further, the term “instruments” or “surgical instruments” or “surgical instrument devices” or “surgical devices” may refer to the singular or plural version of any surgical instruments or tool or accessory not mentioned in the listing above which may be adapted with coatings or other preparations noted herein in order to visualize such instrument, accessory or tool before, during, or following a surgical procedure.

### Detection of Tagged Surgical Instruments

In some embodiments, a surgical instrument is detected in an individual or a subject during surgery with the aid of an imaging technique. Detection of a surgical instrument is achieved via imaging of a tag on or in the surgical instrument, or via imaging a tag that is used in conjunction with the surgical instrument. In some embodiments, a surgical instrument is detected in a subject during surgery in real-time (i.e. during the surgical procedure) using an imaging device that detects the tag. In some embodiments, a surgical instrument is detected in a subject after surgery or after a surgical procedure using an imaging device that detects the tag.

Tags Comprising Imaging Spheres and Use with Imaging Techniques

Disclosed herein is a tag for use with an imaging technique or an imaging device to detect a surgical instrument, wherein the tag comprises an imaging agent. In some embodiments, a tag comprises a matrix and an imaging sphere. In some embodiments, a tag comprises a matrix, imaging sphere, and support medium. In some embodiments, a tag comprises an imaging sphere suspended in a matrix. In some embodiments, a tag comprises an imaging sphere dispersed within a matrix. In some embodiments, a tag comprises an imaging sphere suspended in a matrix and adsorbed to a support medium.

In some embodiments, a matrix comprises any suitable polymer. In some embodiments, the matrix comprises an adhesive polymer. In some embodiments, the matrix comprises a shelf-stable polymer. In some embodiments, the matrix comprises a polymer that is stable to sterilization conditions. In some embodiments, the matrix comprises a polymer that is stable in water, at high temperature, or both.

In some embodiments, the matrix comprises an epoxy. In some embodiments, the matrix comprises a heat-resistant epoxy. In some embodiments, the matrix comprises a water-resistant epoxy. In some embodiments, the matrix comprises a glue, for non-limiting example, cyanoacrylate or acrylic glue. In some embodiments, the matrix comprises an adhesive polymer.

In some embodiments, the imaging sphere comprises a diameter anywhere from the nanometer range to micrometer range or to the sub-millimeter range. In some embodiments, the imaging sphere is visible to the naked eye. In some embodiments, an imaging sphere comprises a diameter ranging in size from about 40 nanometers to about 500 micrometers (micron); from about 40 nanometers to about 300 micron; from about 100 nanometers to about 100 micron; from about 200 nanometers to about 1 micron; from about 200 nanometers to about 700 nanometers; or from about 400 nanometers to about 500 nanometers.

In some embodiments, the imaging sphere is suspended in a matrix in clusters. In some embodiments, multiplied effects are displayed in clusters inside the glue.

In some embodiments, the imaging sphere comprises a hollow cavity and a shell. In some embodiments, subsequent to forming the hollow cavity, such cavity is loaded with an imaging agent (and thereafter is no longer hollow). In some embodiments, the shell comprises inorganic materials (hard shell). In some embodiments, the shell comprises organic materials (soft shell) such as proteins (for non-limiting example, albumin), lipids, or polysaccharides. In some embodiments, a hard shell comprises a polymer, for non-limiting example, cyanoacrylate. In some embodiments, a hard shell comprises silica or silica derivatives. In some embodiments, a hard shell comprises titanium or titanium derivatives, for non-limiting example, titanium dioxide. In some embodiments, a titanium sphere is prepared using titanium t-butoxide.

In some embodiments, a physical characteristic of the imaging sphere's shell is adjusted or optimized by adding a dopant. In some embodiments, a suitable dopant increases porosity of the sphere's shell. In some embodiments, a suitable dopant decreases porosity of the sphere's shell. In some embodiments, a suitable dopant increases the strength of the sphere's shell. In some embodiments, a suitable dopant decreases the strength of the sphere's shell. In some embodiments, a suitable dopant increases the biodegradability of a sphere's shell. In some embodiments, a suitable dopant decreases the biodegradability of a sphere's shell. In some embodiments, the imaging sphere's shell is doped with Iron Oxide or Boron.

In some embodiments, porosity of the imaging sphere's shell is varied by adjusting parameters including electrolyte composition, dopant type, dopant concentration, applied voltage, temperature, light intensity, or any combination thereof

In some embodiments, the porosity of the imaging sphere's shell is directly proportional to the imaging sphere's biodegradability, i.e., an increase in a shell's porosity leads to an increase in the shell's biodegradability. In some embodiments, the porosity of the imaging sphere's shell affects the shelf-life of the imaging sphere. In some embodiments, the porosity of the imaging sphere's shell is inversely proportional to the imaging sphere's shelf-life, i.e., a decrease in a shell's porosity leads to an increase in the shell's shelf-life stability. In some embodiments, the biodegradability of the imaging sphere's shell is preferably optimized via doping. In some embodiments, the shelf-life stability of the imaging sphere's shell is preferably optimized via doping.

Smaller spheres have higher surface area to volume ratios than larger spheres. In certain embodiments, it is advantageous to have spheres with a high surface area to volume ratio. In some embodiments, it is advantageous to have spheres with a low surface area to volume ratio. A wide range of surface to volume ratios are contemplated herein depending on the size of the surgical instrument. For example a small surgical instrument, such as a surgical needle, may require smaller spheres, however, may require a higher total number of spheres in the tag itself to be visualized by the imaging technique. In contrast a larger surgical instrument may only require larger spheres and fewer of them in order to be detectable, although smaller spheres may also work in such an embodiment. In either instance, whether for a large surgical instrument or a smaller surgical instrument, however, nanospheres or spheres of larger-than-nano size may be useful and capable of being detected in tags described herein.

In some embodiments, the hollow cavity of the imaging sphere is loaded (or filled) with an imaging agent. In some embodiments, an imaging agent is loaded into the cavity of the imaging sphere. Thus, provided herein is a sphere comprising a shell defining a cavity, and an imaging agent in said cavity. In some embodiments, the imaging sphere is a nanosphere that encapsulates an imaging agent. In some embodiments, the imaging agent is a gas. In some embodiments, the imaging agent is a gel. In some embodiments, the imaging agent is a hydrogel. In some embodiments, an imaging agent comprises any material that is capable of detection by imaging. In some embodiments, an imaging agent is compatible with one or more of the following imaging techniques: fluoroscopy, magnetic resonance imaging (MRI), ultrasound, X-ray, positron emission tomography (PET), or SPECT tomography. Other imaging techniques compatible with the imaging spheres described herein or any alternative imaging sphere that would be obvious to one of skill in the art upon reading the disclosure herein are considered part of this disclosure and covered by the descriptions herein.

In some embodiments, the imaging agent is compatible with fluoroscopy. In some embodiments, the imaging agent comprises a contrast media, such as barium or iodine. In some embodiments, the imaging agent comprises a radio-opaque contrast media, such as barium. In some embodiments, the imaging agent comprises a radioactive material such as radioactive isotopes. Radioactive imaging agents are compatible with magnetic resonance imaging. In some embodiments, the imaging agent comprises a short-lived isotope. In some embodiments, the short-lived isotope facilitates imaging with gamma cameras. Gamma cameras are used in scintigraphy, SPECT and PET. In some embodiments wherein the imaging technique is SPECT, the short-lived isotope comprises Thallium 201TI, Technetium 99mTC, Iodine 1231, or Gallium 67Ga.

In some embodiments wherein the imaging technique is PET, the imaging agent comprises a short-lived positron emitting isotope, such as ¹⁸F, incorporated into an organic substance.

In some embodiments, the imaging agent comprises echogenic properties. In some embodiments, the imaging agent is detectable via ultrasound. In some embodiments, the imaging agent is detectable via ultrasonic color Doppler imaging. In some embodiments, the imaging agent comprises air, nitrogen, perfluorocarbon, perflutren, fluorocarbon or fluorocarbon-based agents, perfluoropentane, or a combination thereof In some embodiments, the imaging sphere comprises gas that resonates when impinged upon by ultrasound. In some embodiments, the shell of the imaging sphere encapsulates a gas that resonates when impinged upon by ultrasound. In some embodiments where the imaging sphere is a nanosphere that encapsulates a gas that resonates when impinged upon by ultrasound, the imaging sphere is alternatively referred to as a nanobubble. In some embodiments where the imaging sphere is a microsphere that encapsulates a gas that resonates when impinged upon by ultrasound, the imaging sphere is alternatively referred to as a microbubble. In some embodiments, the imaging agent comprises a gas that resonates when impinged upon by ultrasound and emits ultrasound at harmonic frequencies.

In some embodiments, the imaging sphere encapsulates a gas that resonates when impinged upon by ultrasound wherein the gas is fully or partially dissolved in another medium, for non-limiting example, liquid. In some embodiments, the imaging sphere encapsulates a solution saturated with a gas. In some embodiments, the imaging sphere encapsulates a solution saturated with a gas, wherein the solution is shelf-stable. In some embodiments, the imaging sphere encapsulates a solution saturated with a gas, wherein the solution is stable to a variety of temperatures. In some embodiments, the imaging sphere comprises a gas wherein the imaging sphere is shelf-stable. In some embodiments, the imaging sphere comprises a gas wherein the imaging sphere is stable at a variety of temperatures. In some embodiments, the imaging sphere comprises a gas in solution wherein the solution is stable to typical storage conditions, e.g., temperature and length of time.

In some embodiments, the imaging sphere comprises a quantum dot, e.g., CdSe, CdTe, CdS, ZnSe, PbS, and PbTe. In some embodiments, the quantum dot provides a bright fluorescence. In some embodiments, the quantum dot possesses photostability. In some embodiments, the quantum dot exhibits near-infrared emission.

In some embodiments, imaging agents comprise magnetic nanoparticles. In some embodiments, imaging agents comprise superparamagnetic properties. In some embodiments, magnetic nanoparticles are used in conjunction with MRI.

In some embodiments, imaging agents comprise Lanthanide nanoparticles. In some embodiments, Lanthanide nanoparticles are detectable via fluorescence imaging.

In some embodiments of the silica hollow sphere, the silica is doped with dye. Dye-doped silica nanoparticles produce a highly amplified optic signal. In some embodiments, the dye is trapped inside the silica matrix, which provides an effective barrier keeping the dye from the surrounding environment.

### Preparation of Imaging Spheres

In some embodiments, methods for synthesizing an imaging sphere are provided. In some embodiments, a multi-part chemical process for preparing an imaging sphere comprises the following steps: (1) an organic or inorganic material is deposited onto the surface of a bead to form a solid sphere; (2) the core of the bead is removed to create a hollow sphere; and (3) the hollow core is loaded with an imaging agent to form an imaging sphere. In some embodiments, the organic material deposited onto the surface of a bead comprises a protein (e.g., albumin), lipid, or polysaccharide and results in the formation of a “soft shell” around the core. In some embodiments, the inorganic material deposited onto the surface comprises silica or silica derivatives and results in the formation of a “hard shell.” In some embodiments, inorganic material deposited onto the surface of a bead comprises titanium or titanium derivatives, e.g., titanium dioxide, and results in the formation of a “hard shell.” In some embodiments, a hard shell is a silica shell. In some embodiments, the inorganic or organic material is doped with a suitable dopant to adjust the porosity, strength, or biodegradability of the shell.

In some embodiments, the bead is a polymeric bead. In some embodiments, polymer beads are comprised of any suitable polymer. Examples of suitable polymers include, but are not limited to, o-polyacrylamine, poly(vinyl chloride), poly(vinyl chloride) carboxylated, polystyrene, polypropylene, poly (vinyl chloride-co-vinyl acetate co-vinyl) alcohols, latex, or any combination thereof In some embodiments, the polymeric bead is commercially available. In some embodiments, the polymeric bead is synthesized prior to use. In some embodiments, the polymeric bead comprises materials such as polystyrene or latex. In some embodiments, the polymer bead comprises amine or carboxylated derivatives of a polymer.

In some embodiments, polymer beads are commercially available in the desired size. In some embodiments, a polymer bead is synthesized to be a particular size. Polymer beads, for non-limiting example, polystyrene beads, are available commercially in a wide variety of sizes, including beads ranging in size from 45 to 500 nanometers. In some embodiments, the polymer beads are synthesized via emulsion polymerization, which allows for the creation of beads with a particular diameter.

In some embodiments, the polymer core is removed by calcination or solvent extraction resulting in a hollow sphere. In some embodiments, removal of the core results in a uniform, stable silica shell. In some embodiments, removal of the core results in a shell surrounding a hollow cavity.

The shell and/or sphere is biocompatible and safe for use inside a human organism. In some embodiments, the shell and/or sphere is comprised of silica gel and the silica shell and/or silica sphere is biocompatible.

In some embodiments, the hollow sphere is loaded with an imaging agent to form an imaging sphere (sometimes referred to as the “payload”). As used herein, the terms “shell” and “hollow sphere” may be used interchangeably. In some embodiments described herein, the hollow cavity of the sphere is loaded with any imaging agent described herein. In some embodiments, the hollow sphere is prepared prior to loading with an imaging agent. In some embodiments, the hollow sphere is subjected to a vacuum wherein the sphere is evacuated. In some embodiments, the hollow sphere is subjected to a vacuum of 10⁻³ ton. In some embodiments, a vapor comprising an imaging agent is injected into a vessel containing the sphere. In some embodiments, a vapor comprising an imaging agent is injected via a gas syringe into a vessel containing the sphere. In some embodiments, a vapor comprising an imaging agent is injected more than once into the vessel. In some embodiments, degassed water is added to the vessel containing the spheres following injection with the vapor.

In some embodiments, hollow shells and/or loaded shells are produced by one of skill in the art upon reading the present description herein and one or more of U.S. Pat. No. 8,440,229, U.S. Patent App. 20130230570 International Patent App. WO2014052911, and International Patent App. WO2009023697, incorporated herein by reference in their entirety.

Although the majority of the description herein uses spheres as an embodiment of the imaged element in the tag, other three-dimensional shapes are contemplated and covered herein and any reference to sphere or nanosphere herein, whether hollow or not, may alternatively described using non-spherical shapes and nearly-spherical shapes such as, for non-limiting example: ellipsoids, ovoids, spheroids, hyperboloids, paraboloids, cones, cylinders, cubes, cuboids, columns, prisms, rectangular prisms, triangular prisms, hexagonal prisms, pentagonal prisms, octahedrons, dodecaedrons, icosahedrons, pyramids, tetrahedrons, square pyramids, hexagonal pyramids, or any partial three-dimensional version thereof

Loading the Sphere with a Metallic Particle or Metal-Containing Particle

In some embodiments, a sphere is loaded with a metal particle or metal-containing particle. In some embodiments, the metal particle or metal-containing particle is incorporated into the sphere prior to the deposition of the silica or titanium. In some embodiments, an aqueous colloidal suspension of a metal oxide particle precursor or metal oxide nanoparticle precursor is added to a polystyrene composition prior to the deposition of silica or titanium.

In some embodiments, a polymer bead is synthesized using a polymer that comprises a metal oxide particle precursor or metal oxide nanoparticle precursor. In some embodiments, in the synthesis of the sphere, the polymer core is removed while leaving the metallic particle inside the core of the sphere.

In some embodiments, loaded spheres and/or loaded shells are produced by one of skill in the art upon reading the present description herein and one or more of U.S. Pat. No. 8,440,229, U.S. Patent App. 20130230570 International Patent App. WO2014052911, and International Patent App. WO2009023697, incorporated herein by reference in their entirety.

### Spheres Wherein the Surface is Functionalized With an Imaging Agent

In some embodiments, the outer or inner surface of the shell is functionalized with an imaging agent. In some embodiments, the hollow core is additionally loaded with an imaging agent or the same imaging agent that is on the outer or inner surface of the shell. In some embodiments, the outer or inner surface of the shell is functionalized with a fluorophore molecule.

### Surgical Instruments

This novel method of tagging surgical instruments and materials is applicable to a wide variety of surgical instruments. One skilled in the art recognizes that surgical instruments are subject to a variety of naming conventions, often resulting in one surgical instrument referred to by multiple names. Where a surgical instrument is given a particular name herein, one of skill in the art will recognize where that instrument is also known by other names.

Surgical instruments are designed to perform either diagnostic or therapeutic operations to locate the cause of a problem, or to treat a problem once it has been found. Each of the tens of thousands of surgical instruments is designed to perform a specific function. Those functions are generally categorized under one of the following uses: to cut or incise; to retract; to grasp, hold or occlude; to dilate or probe; to cannulate or drain; to aspirate, inject, or infuse; to suture or ligate.

A surgical instrument used according to the methods and included in the devices and systems herein may include a surgical needle. The surgical needle may be straight, ¼ circle, ⅜ circle, ½ circle (subtypes of this needle shape include, from larger to smaller size, CT, CT-1, CT-2 and CT-3), ⅝ circle, compound curve, half curved (also known as ski), and/or half curved at both ends of a straight segment (also known as canoe). The surgical needle may have a particular point geometry, such as a taper geometry (needle body is round and tapers smoothly to a point), a cutting geometry (needle body is triangular and has a sharpened cutting edge on the inside curve), a reverse cutting geometry (cutting edge on the outside), a trocar point or tapercut geometry (needle body is round and tapered, but ends in a small triangular cutting point), a blunt point for sewing friable tissues, a side cutting or spatula point (flat on top and bottom with a cutting edge along the front to one side) for eye surgery. The surgical needle may be an atraumatic needle that is permanently swaged to the suture or may be designed to come off the suture with a sharp straight tug. These “pop-offs” are commonly used for interrupted sutures, where each suture is only passed once and then tied.

In some embodiments, the device, system or method comprises a surgical instrument used to cut or incise, which are frequently referred to as “sharps,” and include scissors, knives, scalpels, chisels and osteotomes, among others. One skilled in the art is able to identify an instrument that is usable for cutting or incising.

In some embodiments, the device, system or method comprises a surgical instrument used to retract, and are frequently referred to as “retractors.” Surgical instruments used to retract include hand-held and self-retaining retractors, as well as instruments such as skin and bone hooks.

In some embodiments, the device, system or method comprises a surgical instrument used to grasp, hold or occlude, and are frequently referred to as “forceps.” Forceps include hemostats, dressing and tissue forceps, bone holding forceps, and needle holders.

In some embodiments, the device, system or method comprises a surgical instrument used to cannulate or drain, such as a catheter, drain, or cannula, for non-limiting example.

In some embodiments, the device, system or method comprises a surgical instrument used to aspirate, inject or infuse, such as a syringe, a needle, a trocar and a cannula, for non-limiting example.

In some embodiments, the device, system or method comprises a surgical instrument used to suture or ligate, such as a suture, clip, suture needle and a ligating instrument.

Surgical instruments (referred to in plural, but which are intended as their singular version in the devices, systems, and methods herein, as appropriate) include, but are not limited to, microsurgical instruments, sponges, towels, scissors, forceps, needle holders, needles, retractors, elevators, awls, bone hooks, bone curettes, osteotomes, chisels, gouges, mallets, tamps, bone files, rasps, bone cutters, trephines, bone rongeurs, bone saws, bone knives, spinal rongeurs, wire implants, pin implants, wire cutters, pin cutters, hand drills, bone screws, bone plates, drill bits, taps, depth gauges, drill sleeves, screwdrivers, bending templates, and plate blending instruments.

In some embodiments, a surgical instrument comprises a particular joint type, handle type, retaining system, blade curvature type, blade type, or bone holding type. In some embodiments, a joint type is a box lock, lap joint or double-action joint. In some embodiments, the handle type is chosen from the following: ring handle, ring handle with one extra large handle, grooved handle, grooved handle with horn, or hollow handle.

In some embodiments, a surgical instrument comprises a retaining system chosen from one of the following: ratchet lock, single spring, double spring, spring with roller, double spring with ball and socket joint, double leaf spring, cam ratchet, bar and wingnut, sliding ring, or bar ratchet.

In some embodiments, a surgical instrument comprises a blade curvature chosen from one of the following: straight, curved on flat, curved on flat-slightly curved, curved on flat-strongly curved, curved on flat- s-shaped, laterally curved, laterally angled, angled on flat, bayonet-shaped, bayonet tip, blunt, blunt with bevel, sharp blunt, angled on flat, sharp, fine tip-sharp, fine tip-blunt, sharp with ball end, blunt with round probe end, blunt with retaining hook, blunt with probe end, blunt and angled on flat, blunt with spade probe end, blunt with triangular section, serrated dissector end, blunt with one hook end, fine straight jaw, round jaw, square jaw, straight or angled on flat, concave cutting jaw, end-cutting straight jaw, end-cutting concave jaw, punch upward, through cutting, punch upward oblique, and not through cutting.

In some embodiments, a surgical instrument comprises a bone holding jaw. In some embodiments, the bone holding jaw is chosen from one of the following: bone holding, bone holding semb, bone holding farabeuf, and bone holding langenbeck.

### Materials Used to Manufacture the Surgical Instrument Base

In some embodiments, a surgical instrument is manufactured from a variety of materials. In some embodiments, instruments are manufactured from stainless steel, tungsten carbide, aluminum, plastics, titanium, or a combination thereof In some embodiments, a surgical instrument is manufactured from stainless steel, which is composed primarily of iron but further comprises one or more of the following elements: carbon; silicon; manganese; phosphorous; sulphur; and chromium.

In some embodiments, a surgical instrument is manufactured from tungsten carbide, which is an alloy of tungsten and carbon. In some embodiments, a surgical instrument is manufactured from aluminum. In some embodiments, a surgical instrument is manufactured from aluminum which is anodized to form an oxide layer on the surface of the aluminum.

In some embodiments, a surgical instrument is manufactured from a biocompatible material such as titanium. In some embodiments, a surgical instrument is manufactured from titanium where the light weight of the titanium prevents surgeon fatigue.

### Methods for Tagging a Surgical Instrument

Methods for Tagging the Surgical Instrument with the Imaging Spheres

In some embodiments, a tag comprising a polymeric matrix and one or more imaging spheres is applied to a surgical instrument as a coating. In some embodiments, a tag comprising one or more imaging spheres suspended in an adhesive polymer is applied to a surgical instrument as a coating. In some embodiments, a tag comprising one or more imaging spheres suspended in an adhesive polymer is applied to a discrete portion of a surgical instrument. In some embodiments, a tag comprising one or more imaging spheres suspended in an adhesive polymer is applied to a surgical instrument, wherein the tag is a small shape, e.g., dot, square, stripe. In some embodiments, a tag comprising one or more imaging spheres suspended in an epoxy is applied to a surgical instrument, for non-limiting example, a needle.

In some embodiments, a tag comprising a mixture of a polymer and one or more imaging spheres is applied to a surgical instrument. In some embodiments, a tag comprising a mixture of an adhesive polymer and one or more imaging spheres is applied to a surgical instrument, e.g., needle. In some embodiments, a tag comprising a mixture of an epoxy and one or more imaging spheres is applied to a surgical instrument, for non-limiting example, a needle.

In some embodiments, a tag comprising one or more imaging spheres suspended in a polymeric matrix and adhered to the surface of a support medium is applied to a surgical instrument. In some embodiments, a tag comprising one or more imaging spheres suspended in a polymeric matrix and adhered to the surface of a support medium is applied to a surgical instrument via an adhesive polymer on the support medium. In some embodiments, a tag comprising one or more imaging spheres suspended in an adhesive polymer and adhered to the surface of a support medium is applied to a surgical instrument. In some embodiments, a tag comprising one or more imaging spheres suspended in an epoxy and adhered to the surface of a support medium is applied to a surgical instrument.

In some embodiments, a separate coating is applied to a tagged surgical instrument to prevent degradation of the tag during sterilization.

In some embodiments, a tag comprising one or more imaging spheres suspended in a polymeric matrix is embedded in a surgical instrument. In some embodiments, a tag comprising one or more imaging spheres suspended in a polymeric matrix is embedded into a thread bundle of a needle. In some embodiments, a tag comprising one or more imaging spheres suspended in an adhesive polymer is embedded in a surgical instrument. In some embodiments, a tag comprising one or more imaging spheres suspended in an epoxy is embedded in a surgical instrument. In some embodiments, a tag comprising imaging spheres suspended in an adhesive polymer is embedded into a thread bundle of a needle. In some embodiments, a tag comprising imaging spheres suspended in epoxy is embedded into a thread filament or thread bundle or thread in a surgical instrument, e.g., needle.

### EXAMPLES

The present invention may be better understood through reference to the following non-limiting examples. These examples are included to describe exemplary embodiments only and should not be interpreted to encompass the entire breadth of the invention.

### Example 1

### Tagging a Surgical Needle

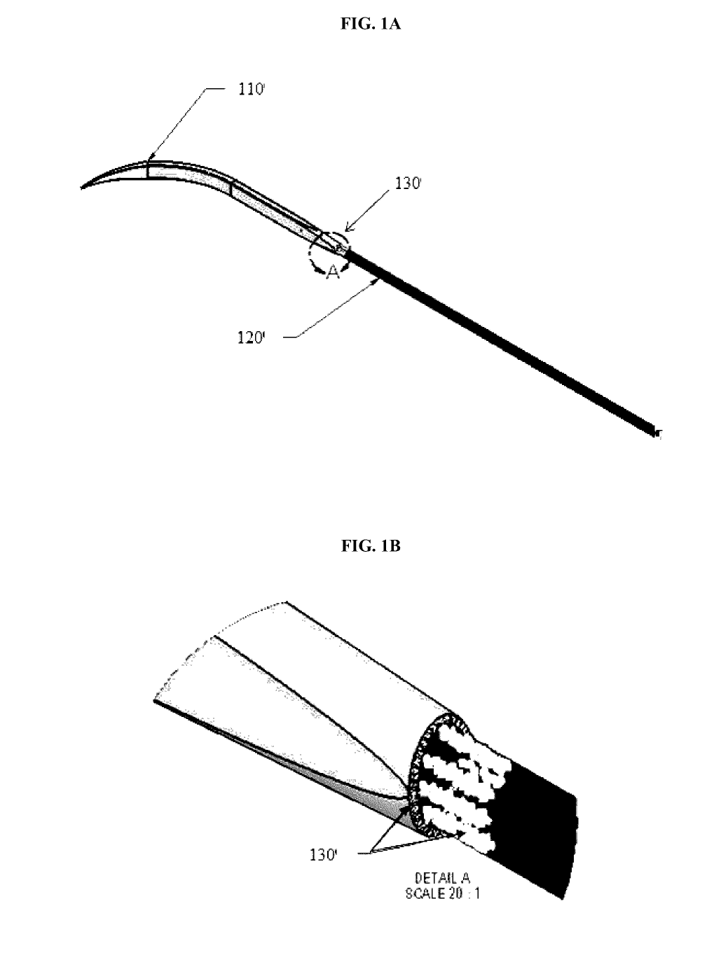

FIG. 1 illustrates a tagged needle. FIG. 1A depicts an embodiment including a surgical instrument comprising a surgical needle 110, a surgical thread 120, and a tag 130 comprising an imaging sphere suspended in an adhesive polymer placed at the joint area of the surgical needle and surgical thread. FIG. 1B depicts a magnified view of the joint area of the surgical instrument embodiment of FIG. 1A. To prepare the tagged needle in FIG. 1A and 1B, a tag is prepared as follows. A commercially available 2.0 micron polystyrene bead is used. The polystyrene bead is coated with a polyamine to facilitate a sol gel reaction. The bead is mixed with tetramethylorthosilicate for 2 hours, followed by addition of trimethoxyboron. The resulting particles are washed with water and ethanol to remove unreacted starting materials. The polystyrene bead core is subsequently removed via calcination at 550° C. for 18 hours. This yields a hollow porous silica gel particle with uniform wall thickness of approximately 10 nm. The hollow silica particles are then loaded with perfluoropentane by injecting PFP vapors using a gas syringe into a vessel containing the hollow silica particles. The injection of PFP vapors is performed 4 times. The resultant gas-filled silica spheres are sonicated to create a uniform suspension. The silica spheres are then mixed with cyanoacrylate to form an adhesive polymer with imaging properties. The resultant adhesive polymer is coated onto a thread filament in the joint area between the surgical needle and the surgical thread. Additionally or alternatively, the resultant adhesive polymer is used to adhere the thread filament to the surgical needle.

All publications, patents, and patent applications mentioned in this specification are herein incorporated by reference to the same extent as if each individual publication, patent, or patent application was specifically and individually indicated to be incorporated by reference. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims, at least), define the scope of the invention and that methods and structures within the scope of these claims and their equivalents be covered thereby.

1. A tag to mark instruments, the tag comprising:
a matrix; and
a plurality of imaging nanospheres suspended within the matrix, the imaging nanospheres each having a respective shell that forms a respective cavity, each of the cavities loaded with an imaging agent that enhances a visibility of the respective imaging sphere in an image.

2. (canceled)

3. The tag of claim 1 wherein the matrix comprises an adhesive polymer.

4. The tag of claim 1 wherein the matrix comprises cyanoacrylate.

5. (canceled)

6. The tag of claim 1, wherein the matrix comprises an epoxy.

7. (canceled)

8. The tag of claim 1 wherein the imaging agent is an echogenic, imaging agent.

9. The tag of claim 1 wherein the imaging agent is a gas.

10. The tag of claim 1 wherein the imaging agent is gas is dissolved in a solution.

11. The tag of claim 1 wherein the imaging agent is a gas that resonates at a harmonic frequency when impinged upon by ultrasound.

12. (canceled)

13. The tag of claim 1 wherein the shell comprises an inorganic material.

14. The tag of claim 13 wherein the inorganic material comprises silica or titanium.

15. A tagged surgical instrument, the tagged surgical instrument comprising:
a matrix;
a plurality of imaging nanospheres suspended within the matrix, the imaging nanospheres each having a respective shell that forms a respective cavity, each of the cavities loaded with an imaging agent that enhances a visibility of the respective imaging sphere in an image; and
a surgical instrument to which the matrix is attached.

16. (canceled)

17. The tagged surgical instrument of claim 15, wherein the surgical instrument is a needle, the matrix is embedded in a thread filament, and the matrix attaches the thread filament to the needle.

18. (canceled)

19. A method, the method comprising:
depositing organic or inorganic material onto a plurality of bead surfaces to form a plurality of nanospheres each with a solid core;
removing the solid core of each of the nanospheres by solvent extraction or calcination to form a plurality of hollow nanospheres;
loading the hollow nanospheres with an imaging agent that enhances a visibility of the hollow nanospheres in an image; and
suspending the plurality of hollow nanospheres loaded with the imaging agent in a matrix.

20-21. (canceled)

22. The method of claim 19 wherein depositing organic or inorganic material onto a plurality of bead surfaces includes depositing silica or titanium onto a plurality of thamine-functionalized polystyrene beads.

23-25. (canceled)

26. The method of claim 19 wherein depositing organic or inorganic material onto a plurality of bead surfaces includes depositing the organic or inorganic material onto a plurality of bead surfaces each having a diameter between about 500 nm to about 10 micron.

27. (canceled)

28. The method of claim 19 wherein depositing organic or inorganic material onto a plurality of bead surfaces includes depositing a doped organic or inorganic material onto the plurality of bead surfaces, the doped organic or inorganic material comprising a dopant.

29. The method of claim 1 wherein depositing organic or inorganic material onto a plurality of bead surfaces includes depositing a doped organic or inorganic material onto the plurality of bead surfaces, the doped organic or inorganic material comprising a dopant that comprises Iron(III) Oxide or Boron.

30. The method of claim 19, further comprising:
subjecting the hollow nanospheres to a vacuum prior to loading the hollow nanospheres with the imaging agent.

31. The method of claim 19 wherein loading the hollow nanospheres with an imaging agent includes loading the hollow nanospheres with an echogenic imaging agent.

32. The method of claim 19 wherein loading the hollow nanospheres with an imaging agent includes loading the hollow nanospheres with a gas.

33. The method of claim 19 wherein loading the hollow nanospheres with an imaging agent includes loading the hollow nanospheres with a gas dissolved in a solution.

34. The method of claim 19 wherein loading the hollow nanospheres with an imaging agent includes loading the hollow nanospheres with a gas that resonates at harmonic frequencies when impinged upon by ultrasound.

35-37. (canceled)

38. The method of claim 19, further comprising:
applying the matrix, to a portion of an instrument.

39. (canceled)
